(19) United States
(12) Patent Application Publication
(10) Pub. No.: US 2020/0229707 A1
(43) Pub. Date: Jul. 23, 2020

(54) Device and Method for Equine Condition Monitoring

(71) Applicant: Sandra DONNELLY, Trabuce Canyon, CA (US)

(72) Inventor: Sandra Donnelly, Trabuca Canyon, CA (US)

(21) Appl. No.: 15/754,991

(22) PCT Filed: Aug. 24, 2016
(86) PCT No.: PCT/US2016/048484

§ 371 Date: Feb. 23, 2018

Related U.S. Application Data
(60) Provisional application No. 62/209,190, filed on Aug. 24, 2015.

(51) Int. Cl. A61B 5/0205; A61B 5/00; A61B 5/01

(52) U.S. Cl. CPC A61B 5/0205; A61B 5/7435; A61B 5/0004; A61B 5/0024; A01K 29/005; A61B 5/4277; A61B 5/682; A61B 2503/40; A61B 5/01

(57) ABSTRACT

An equine condition monitoring device and method is provided which employs one or more sensors operatively engaged upon the body of a horse during exercise, to gather and store electronic signals correlating to physiological characteristics of the horse during exercise. The electronic signals may be input to software to provide graphic depictions such as video displays or printed depictions showing the physiological characteristics being monitored at any point in time for the duration of exercise of a horse.

This application claims the benefit of U.S. Provisional Application No. 62/209,190 filed on Aug. 24, 2015 and is incorporated herein in its entirety.

### BACKGROUND OF THE INVENTION

### 1. Field of the Invention

The present invention relates to monitoring oxygen levels, body temperature, pulse rate and other physiological or health indicators. More particularly, it relates to a device enabling a method for real time monitoring of the current physical condition for horses and other animals using a device located within their mouth configured for sensing and communicating information concerning blood oxygen levels, body temperature, heart rate, breathing and other health factors which can be sensed electronically.

### 2. Prior Art

Horses which engage in equine sports, such as horse racing or jumping, are athletes which as is well known, benefit with increased performance after training for their respective sport. Equine training involves subjecting the animal to regular periods of exercise in a manner calculated to promote changes in the structure and function of the animal in order to enable the animal such as a horse, to compete more effectively.

As a result of regular training, adaptations occur in the cardiovascular system, muscle cells, and in other tissues such as tendons and bones. The response of the animal physically, is a direct result which is dependent on the use of appropriate training depending upon the current condition of the animal, and the goal for conditioning and the time available for such training to reach the physical goal in mind for the horse.

A common issue during horse training is the potential for over-exercising a horse during training. Essentially such occurs where horses have had a sudden increase in training speeds or distances, or have been entered in competition before they are fully prepared. The excess increase in exercise can cause a sudden increase in stress on bone, tendons and other structures which can result in injury. Further, the respiratory system of horses are prone to bleeding caused by burst blood vessels in the lungs which can easily occur during excessive exercise for the current physical condition of a horse.

Consequently, it is most important to be able to monitor the psychological factors of a horse or other animal during training, to ascertain if the horse is being over-exercised, or under-exercised, and to determine the subsequent length and nature of training periods for the animal. One method employed by trainers is the use of a treadmill and monitoring of the blood oxygen levels of the horse during a session. Other factors such as heart rate, breathing rate, and temperature may be monitored also.

However, training on a treadmill while delivering measurable and variable exercise to the horse, is not training under real world conditions for the sport in which the horse may be competing. Racehorses require strength and stamina over varying race courses on which they may be competing. Such courses vary widely in distance, climate, race surface, and other factors which cannot be simulated on a treadmill.

The same holds true of runner-jumper horses and other horses competing in differing equine sports. Training using real-world situations for upcoming events helps the horse physically in working the muscles, legs, as well as the cardiovascular and respiratory systems under real world conditions.

However, monitoring physiological factors of the body and horse health in real world conditions is a widespread problem in equine training. While using sensors to monitor blood oxygen levels, respiration, heart rate, temperature and the like are available where the animal is essentially stationary on a treadmill, such is not the case for horses training on a real racetrack or jumping course. Thus, the trainer is left with taking data measurements before and after the training exercise to determine training progress.

As such, there is an unmet need for a device and method which may be employed to continuously monitor physiological factors of an animal such as a horse which can be employed both on a treadmill or other stationary exerciser, and upon a course on which the animal is trained under race or competition conditions. Such a device would allow trainers to view data concerning oxygen levels, body temperature, respiration, and other physiological aspects which can be captured by sensors in real time, and communicated to the trainer in relation to time and duration of the exercises, and the position of the animal on the course.

Still further, in a particularly preferred mode of the device and method, such a device should in real time, using GPS cellular triangulation or other wireless geographic position sensing means, correlate the animals actual position on a course or track with the current data concerning physiological conditions of the horse such as oxygen level, respiratory factors, temperature, heart rate and the like. In doing so, trainers can ascertain patterns in the training in real time and position on a track or course where the animal begins to become fatigued or where they get a second-wind and adapt future training sessions to adjust the regimen, taking such into consideration.

Finally, in a particularly preferred version of the system herein, using global positioning such as GPS and commercially available mapping systems such as GOOGLE EARTH, the system can provide video depictions of each track and course viewable on a video display, which correlates the measured physiological condition of the animal at multiple positions on the course. Such will provide a visual depiction of horse conditioning and using captured GPS positioning of the device engaged to the horse during a training exercise, correlate the various sensed physiological measurements of horse conditioning to the ascertained GPS position of the animal, for automatic loading of the data into a video depiction of the actual course or track on which the training occurs.

### SUMMARY OF THE INVENTION

The device and method herein disclosed and described achieves the above-mentioned goals through the provision of a sensing component adapted for engagement or in combination with a tongue tie which is a piece of equipment used by equestrians to prevent a horse from getting its tongue over the bit, which would make the animal very difficult to control. Tongue ties are usually formed of a flexible member of cloth or synthetic material which is passed through the mouth and secured below the chin. The sensing component may be employed as a unit permanently installed on a tongue tie, or adapted to operatively engage a tongue tie to gather measurable physiological data concerning the current state of the horse.

The sensing component device will have a housing formed of material adapted to survive a fluid environment in the mouth of a horse. The housing preferably has an exterior surface defining a curved shape on at least a first side surface adapted for engagement with the tongue tie which would more comfortably curve over the first side surface.

A second side surface could be curved or planar or combinations thereof adapted to comfortably operatively contact with the surface of the tongue of the horse wearing the device during exercise. Operatively positioned on or adjacent this second surface will be the electronic sensors for sensing oxygen saturation of the blood running through the tissue of the horse against which the second side of the device is positioned. Such oxygen sensors conventionally illuminate the capillaries and sense the reflected or translucent color of the blood running through the capillaries. From the ascertained color compared to a database or software library of colors related to oxygen saturation, the current oxygen saturation of the horse or animal can be determined.

Other sensors for heart rate, breathing, temperature of surrounding tissues, fluid contents of saliva, and any other sensor capable of providing a digital output of a sensed input within the mouth of the animal such as a horse, can also be operatively engaged to the housing. In this fashion, using the device, multiple sensed physiological conditions of the horse can be concurrently ascertained, by the plurality of different sensors. The digital output from the sensors can be stored in electronic memory positioned in the housing, and/or communicated to a microprocessor running software adapted to intake multiple digital data streams from multiple sensors, and output such to electronic memory and/or to a connection such as a USB wired connection, or a wireless communicator such as a transceiver operatively engaged to the microprocessor or sensors. The RF or light-based transceiver can wirelessly communicate the digital electronic feeds from the sensors which are representative of current sensed physiological information such as oxygen saturation, body temperature, respiration rate, and respiration breathing sounds, to a receiving component which may be operatively engaged to a computer having software adapted to receive each digital feed, and output visual depictions of the changing data.

Additionally preferred would be the incorporation of location determination components and software adapted to determine the location of the horse or animal on the earth. Such can employ GPS or cellular triangulation, or using local GPS augmenting signals, can ascertain the location and movement of the horse on a track or training facility, and in real time track the movement. The location information would also be transmitted by the wireless transmitter to a receiver such as the above noted computing device.

Using this location-ascertaining ability and tracking ability, the streaming data can be correlated to commercially available geo-location software such as GOOGLE MAPS, to ascertain the racetrack or training facility or other track on which the horse is moving. As this location and speed information will stream as electronic data along with the sensor electronic data streams, the sensed physiological information about the horse, in real time, at various positions around a track or facility, can be correlated. This will allow the trainer to ascertain the physical condition of the horse, based on the sensed information in real time at exact positions on a track or training facility.

The exact track or training facility can also be determined by taking the GPS or other location data transmitted from the device on the animal and correlating it to positions on the earth. In this fashion, representations of a track or facility may be depicted on a video display, with representations of the position of the animal during training concurrently with indicia relating to the streams of data from multiple sensors broadcast or communicated from the device as the horse exercises or thereafter. Thus, the trainer will be able to ascertain where the horse loses speed on the track and correlate it to the sensed physiological information such as oxygen levels, temperature, respiration, and then adjust the training regimen more accurately.

Communication of the stored electronic data can be done with a wired USB connection or the like, or the wireless transmitter may be included within the housing of the device, or may be in wired communication from a position outside the mouth of the horse where wires to the transmitter will run through the belt of the formed tongue tie. Alternatively, a bluetooth or other wireless transceiver can communicate wirelessly with a repeater located outside the mouth of the horse which can transmit GPS data to the device and to the receiving device for the data streams from the mouth-located device herein.

Locating a repeater or transceiver outside the mouth of the horse may be preferable to more accurately ascertain GPS position and movement, as well as to communicate the sensor data streams in real time since the anatomy of the horse surrounding the device within the mouth can affect accuracy and speed of RF signals.

Mounting the device can be by engagement of the housing to a tongue tie and engagement of the tongue tie to the horse. Alternatively, the housing and device can be made as part of the tongue tie, which would simply be engaged to the horse. Once operatively engaged, a test of the sensors and data streams therefrom can be run, and thereafter the data streams and GPS information stream can be received and correlated to produce the graphic interface showing position on the course or track, and current physiological conditions for the physical conditions sensed by the sensors during the work out.

It is thus an object of this invention to provide a mouth engaged sensing device for current physical condition of a horse which can store or communicate data streams in real time from sensors.

It is a further object of this invention to provide such a sensing device, which can determine location of the horse on the planet and current moving speed, to show positioning of the horse on a track or course correlated with current sensed physical information.

These together with other objects and advantages which become subsequently apparent reside in the details of the construction and operation as more fully hereinafter described and claimed, reference being had to the accompanying drawings forming a part thereof, wherein like numerals refer to like parts throughout.

With respect to the above summary, before explaining at least one preferred embodiment of the herein disclosed horse or animal training monitoring device in detail, it is to be understood that the invention is not limited in its application to the details of construction and to the arrangement of the components in the following description or illustrated in the drawings. The invention herein described is capable of other embodiments and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other components, methods and systems for carrying out the several purposes of the present disclosed device and system herein. It is important, therefore, that the claims be regarded as including such equivalent construction and methodology insofar as they do not depart from the spirit and scope of the present invention.

### BRIEF DESCRIPTION OF DRAWING FIGURES

FIG. 1 depicts a view of an animal such as a horse with a tongue tie secured through the mouth and around the lower jaw.

FIG. 2 depicts a view of the horse of FIG. 1, showing a repeater or secondary transceiver located on a belt, exterior to the mouth of the horse.

FIG. 3 depicts a sectional view of the housing of the sensing device in contact with the tongue of a horse and the tongue tie engaged around the jaw and tongue.

FIG. 4 depicts a sectional view of the housing and sensing device of FIG. 3, adapted for engagement to the tongue of the animal, showing some of the components therein.

FIG. 5 depicts a mode of the device enabling the method herein, adapted for engagement on the tail of a horse.

FIG. 6 depicts one component for monitoring the oxygen levels and pulse rate of the horse or similar animal as would be employed in the device of FIG. 4 or FIG. 7.

FIG. 7 shows a sectional view through a mode of the device adapted for engagement to the underside of the tail at the dock of the horse with components the same as would be employed for the tongue mode of the device of FIG. 4.

FIG. 8 shows a graphic depiction of a track such as on a video display showing the track which may be mapped and monitored by GPS and/or local location beacons which is segmented into positions thereon for cross referencing of data from the animal-engaged device herein, at a point in time the horse occupied a position.

### DETAILED DESCRIPTION OF THE PREFERRED EMBODIMENTS OF THE INVENTION

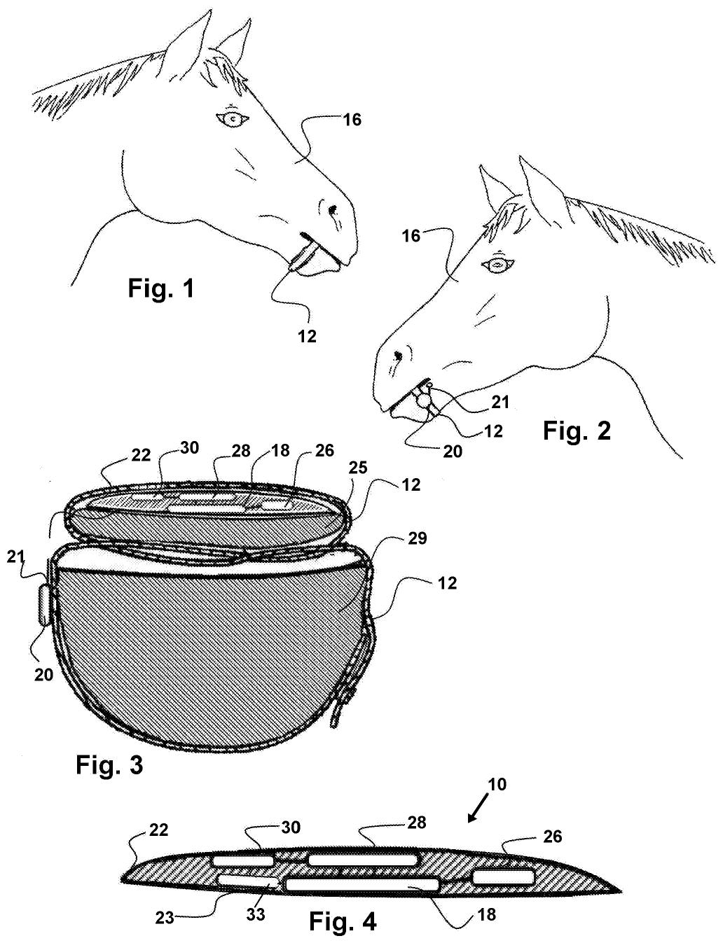

Referring now to the drawings of FIGS. 1-8 there is shown in FIG. 1, a view of the device 10 herein operatively engaged with a tongue tie or belt 12 on a horse, which is secured using a flexible member running through the mouth and around the lower jaw 29. Securement of the flexible member or belt can be by hook and loop fabric or cooperative fasteners allowing for the operative positioning of the device 10 within the mouth of the horse 16 or animal and thereby placing the sensors 18 (FIGS. 3-5) in operative position within the mouth and against the flesh or tongue of the animal. Such sensors 18 can include one or a combination of sensors from a group for sensing physiological characteristics including temperature by thermometers, breathing by microphones, blood oxygen from pulse oximeters, heart rate sensors, fluid content sensors, blood sugar sensors, and other sensors as would occur to those skilled in the art which will output an electronic signal which may be correlated to a particular physiology such as oxygen levels in the blood, body temperature, respiration and the like.

A reverse view of the engagement of FIG. 1 is depicted in FIG. 2, to also show a belt 12 engaged to an exterior antenna 21 or alternatively a secondary wireless repeater or secondary transceiver 20 for Bluetooth or Wifi or Optical or other communication located on a belt or a component of a tongue tie belt 12. If a secondary transceiver 20 is employed, placing it on the exterior of the belt 12 positions the secondary transceiver 20 outside the mouth in a better position to communicate with the transreceiver 30 within the device 10 in the mouth or on the tail 17 of the animal, to which sensory data streams and GPS or location information data is transmitted. A GPS or wireless location-determining component can also be included in the secondary transceiver 20 to place it in better positioning for location signal receipt as well as transmission.

Alternatively, as shown in FIGS. 2 and 7, and preferably in either mode of the device 10 where it is mounted in the mouth or on the tail 17, an antenna 21 is positioned upon the belt and is operatively engaged to the device 10 using a lead 25 running from the housing 22 of the device 10 to the antenna 21. This positions the antenna 21 in a fairly unobstructed position. In experimentation it was found the device 10 performed significantly better as communication from the GPS satellites, or local beacons, was received better, and if real time broadcasting of the oxygen and heart rate is communicated wirelessly to a remote device on the track, the data transmission rate improves with the exterior antenna 21 and connected by the lead 25 running through one of the two flexible members forming the belt. As such, while the device 10 will operate with the antenna engaged upon or within the housing 22, the accuracy of track position determination using GPS or local beacons, and data transmission rate improves using the belt 12 engaged antenna 21, in all modes of the device 10, and such is preferred.

FIG. 3 shows a sectional view of the housing 22 of the sensing device 10 as configured for operative contact with the tongue 24 of a horse 16, which includes the same components as the mode of the device of FIG. 7, adapted for engagement with the tail 17 of the horse 16. As shown in the mode of the device of FIG. 3, a contact side 23 of the housing 22 is configured for direct contact with the skin of the tongue or tail 17 of a horse 16.

As depicted in FIG. 4, the contact side 23 of the housing is generally planar, although recent experimenting, indicates that a contact side 23 of the housing 22, having a recessed central area 27, as in FIG. 7, improves data capture by positioning the sensors 18 centrally, which provides a better sensing contact with the skin of the tail 17 or tongue 19, when the belt 12 is cinched. Experimentation indicates that the curved or recessed central area 27 provides an enhanced contact with the skin by following the natural curve of the tail 17 or underside of the tongue, and such may be preferable.

In the mode of the device 10 of FIGS. 1-3, the housing 22 is engaged with the contact side 23 in a contact with the skin of the tongue, with the belt 12 engaging around the tongue. As shown in FIG. 3, a secondary belt 12 portion, may also be engaged around the lower jaw 29.

The device 10 is held in this operative engagement of sensors 18 against the skin of the tongue, using the noted belt 12, which may be configured as a tongue tie and/or with lower jaw encirclement. Of course other means for holding the device 10 in operative position in skin contact in the mouth may be employed and other positions of the sensors 18 used, when engaged in the mouth.

Shown in FIG. 4 there is a sectional view of the housing 22 of the sensing device 10 engaged to the belt 12 of FIG. 3 as employed in FIGS. 1-2 in operative engagement with the mouth of the horse 16. In this mode, as well as the mode of FIG. 7, the housing 22 of the sensing device 10 is formed of material adapted to survive in a fluid environment in the mouth of the horse 16, or on the tail 17, and in wet or dry conditions on the track.

As shown, in the mode of the device 10 for mouth engagement, the exterior surface of a first side of the housing 22, is shaped to a curve to better accommodate the belt 12 running thereover. However, it may be employed such as the particularly preferred shape of the contact side 23 in FIG. 7 which centrally locates the sensor 18 in a recess. If positioned within the mouth, the curved first side of the housing opposite the contact side 23, is well adapted for engagement with the tongue using a belt 12 which will more comfortably curve over the opposite side surface and leave less of a gap on the side edges.

As noted, the contact side 23 opposite the first side surface may preferably be curved forming a central recessed area 27 as in FIG. 7, or it may be planar or combinations thereof and is configured to operatively contact with the surface of the tongue 19 of the horse 16 wearing the device 10 during exercise and position sensors 18 thereon or adjacent thereto.

In all modes of the device 10, one or a plurality of electronic sensors 18 can be included from a group of electronic sensors configured for sensing physiological characteristics herein, such as sensing oxygen saturation, heart rate, breathing rate, body temperature of surrounding tissues, fluid contents of saliva, and any other electronic sensor capable of providing an electronic or digital signal output, correlated with a sensed input within the mouth or on the tail 17 of the animal such as a horse 16. When employed in the mouth, a microphone might also be included to communicate breathing sounds and rates in a digital data stream.

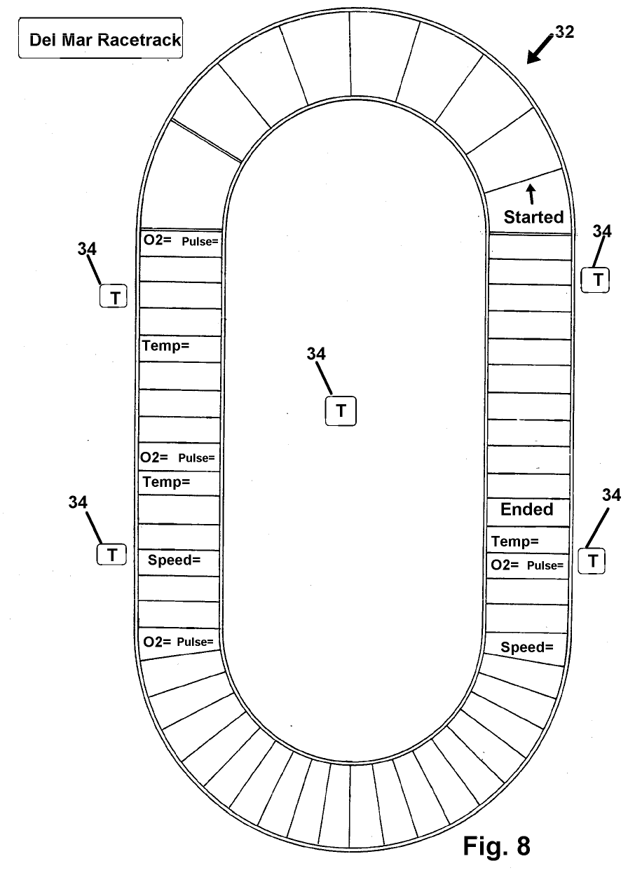

The digital output from the sensors 18, in all modes of the device 10, can be stored in electronic memory 26 and/or communicated to a microprocessor 28 running software adapted to intake multiple digital data stream signals, from multiple sensors 18, and output such to electronic memory 26 and/or to a connection such as a USB connection, or a connection such as wireless communicator such as a wireless transceiver 30. The transceiver 30 can wirelessly communicate the digital electronic signal streams from the sensors 18, to a receiving component which may be operatively engaged to a computer having software adapted to receive each of the digital signal streams, and output visual depictions 32 of the changing data related to the current physical state of the horse 16 such as shown in FIG. 8. Such, for example, may include oxygen saturation and heart rate over the duration of exercise, so that the user may discern the condition of the horse at any time during an exercise or race, related with a position on the track. As noted the connection to offload the data from the signals from the sensors, can also be simply stored in electronic memory, and offloaded with a USB, or serial, or other wired connection (not shown but well known) to the computer running the software to correlated the data.

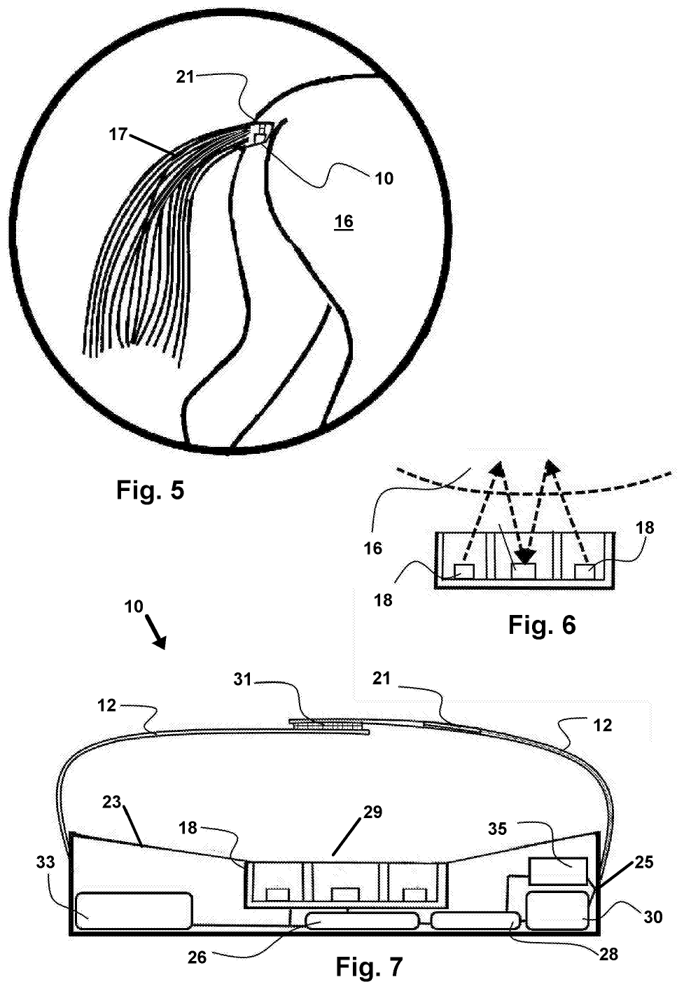

Shown in FIG. 5 is a mode of the device 10 enabling the method herein, showing the housing 22 adapted for operative engagement on the underside of the tail 17 of a horse 16 at or adjacent of the dock. As depicted, the contact side 23 of the housing 22 is in direct contact with the skin of the underside of the tail 17.

As is also shown, the belt 12 is formed by two portions of a flexible member which communicate around the tail 17 to an engagement of distal ends using complimentary fasteners 31 (FIG. 7) to hold the distal ends engaged. This engagement cinches the belt 12 and biases the contact side 23 of the housing 22 against the skin of the underside of the tail 17. It also positions the preferred antenna 21, on a side or upper surface of the tail 17, where it can receive positioning signals from GPS satellites and/or positioning signals from local beacons, which are communicated to the microprocessor 26 or computer which has software running in electronic memory 28 to receive the positioning signals from the antenna 21, and correlate a current position of the horse on a track, with the data concerning the physiology of the horse 16 from the sensors 18.

Depicted in FIG. 6 is an example of one sensor 18 component which may be employed for monitoring the oxygen levels and pulse rate of the horse 16 or similar animal as would be employed in the device of FIG. 4 or FIG. 7. Shown is a reflective optical sensor using a reflective reception of infrared and red LED's to a photo transistor which generates an electric signal which correlates to a current pulse rate and oxygen saturation. Such is available for example from Japan Radio Company in Japan, and can provide a sensor 18 as in FIG. 7 to output to electronic memory 26 and/or the microprocessor 28, correlated to the current pulse and oxygen level of the horse 16. Of course other sensors for sensing health and fitness related horse physiology can be employed such as a thermometer, and any sensor 18 which can provide an electric signal output, correlating with a particular physical condition of the horse, is anticipated within the scope of this patent application.

Shown in FIG. 7 shows a sectional view through a mode of the device 10 which as noted has a housing 22 adapted for engagement to the skin on the underside of the base of the tail 17 at the dock of the horse 16. Shown are components which would also be employable with the mode of the device 10 of FIG. 7 such as a battery 33, the sensor 18, electronic memory 26, a computer or microprocessor 28 for running software held in electronic memory 26, a wireless transmitter or transceiver 30, and a GPS location sensor 35.

As shown in FIG. 7, the transceiver 30 and GPS location sensor 35 may be preferably connected with a lead 25 running along a portion of the belt 12 to an antenna 21 which will be facing upward or outward when the distal ends of the belts 12 are engaged with complimentary fasteners 31 located thereon. The fasteners can be any complimentary fastener 31 suited to the task, such snaps, buttons, hook and loop fabric, hooks, or other fasteners adapted to the task.

FIG. 8 shows a graphic depiction 32 which may be generated on a video display of a representation of a track. In a method for tracking horse health, using the components herein, as noted above, the position of the horse 16 during exercise and training on the track can be ascertained with the GPS receiver 35 or other location sensing components. The electronic data concerning aspects of the physiology of the horse 16, from communicated data steams from each respective sensor 18, when received to electronic memory 26 and the computer or microprocessor 28, can be correlated with a current position of the horse 16 on the track determined by the GPS receiver 35, and depicted in the position on the track where the data was collected from the sensors 18. The data from the sensors 18, such as current blood oxygen levels and pulse rate, can be correlated at any time during the exercise or race, and can also be correlated with the position of the horse 16 on the track.

This method for horse health monitoring can be provided in real time by a broadcast from the transceiver 30 and antenna 21 or can be stored in electronic memory 26 and offloaded upon completion of exercise by the horse 16. If offloaded, a local computing device running software adapted to use the offloaded data, and correlate the physical data from the sensors 18 with the location data from the GPS receiver 35, and produce graphs showing the correlated information, or graphic depictions such as the track depicted in FIG. 8, with segmented positions thereround, for showing current horse condition from the sensors 18. It should be noted, that extra location transmitters 34 can be employed to generate a location signal which can augment or replace the location signal of the GPS receiver 35, which can employ GPS satellite signals or cellular tower location signals. Such location transmitters 34 would transmit signals employable by the GPS receiver 35 to augment the location calculation.

As noted, the streaming data correlated to horse physiology, from the sensors 18 which is provided from the device 10, along with the terrestrial positioning calculated by the GPS receiver during exercise, can be correlated to commercially available geo-location software such as GOOGLE MAPS. In this fashion, a location of a particular racetrack or training facility may be ascertained on which the horse 16 is moving. Thus, the trainer will be able to ascertain where on a current track the horse 16 loses speed, or where the horses performance changes on that track. This allows the device 10 and system herein, to be employed on virtually any track or exercise path, since GOOGLE MAPS or similar location data may be employed to discern the position, start, stop, and speed, on that track, in combination with data stored from the electronic location or GPS receiver 35. Changes in speed or performance, discerned therefrom, can then be correlated to the sensed physiological information from the sensors 18, to better ascertain current horse conditioning and any needed training.

In the method herein, the device 10 is operatively engaged to the tongue or tail 17 of a horse 16. Upon a start of exercise of the horse 16, electronic data from the sensors 18 correlating to the physiology of the horse 18 is communicated to memory for the term of the exercise. Concurrently, electronic signals correlating to location and speed of the horse during the exercise, from the GPS sensors, is also communicated to electronic memory. Concurrently during the horse exercise, or subsequent thereto, the electronic signals correlating to the physiology of the horse, and the electronic signal correlating to the speed and location of the horse, are input to software adapted to the task of graphically depicting the data relating to the horse physiology with the data relating to the horse location and speed, at any point in time during the horse exercise. Using the graphic depiction, the trainer may then adjust the horse exercise regimen accordingly.

While all of the fundamental characteristics and features of the invention have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the spirit or scope of the invention. Consequently, all such modifications and variations and substitutions are considered included within the scope of the invention as defined by the following claims.

What is claimed is:

1. An equine condition monitoring apparatus comprising:
a housing, said housing configured for positioning to an engaged position with an engagement side of said housing, adjacent a portion of skin of a horse;
at least one sensor providing an electric signal output correlating to a physiological characteristics of said horse;
a flexible member engaged with said housing, said flexible member engageable with said horse to maintain said housing in said engaged position;
electronic memory positioned in said housing for storing said electric signal output during a duration of movement of said horse; and
a connection to communicate said electric signal stored in said electronic memory, to a remote computing device, whereby software adapted to employ said electric signal correlates said physiological characteristic represented by said electronic signal to a time of said duration of movement of said horse, and outputs a graphic depiction thereof.

2. The equine condition monitoring apparatus of claim 1, additionally comprising:
a terrestrial location sensor engaged with said housing;
said terrestrial location sensor outputting a location signal to said electronic memory; and
said software adapted to employ said electric signal is further adapted to employ said location signal, and correlate a position of said horse during said time of said duration of movement to said graphic depiction.

3. The equine condition monitoring apparatus of claim 1, additional comprising:
said connection to communicate said electric signal stored in said electronic comprises a transceiver engaged with said housing; and
said transceiver wirelessly communicating with said remote communicating device.

4. The equine condition monitoring apparatus of claim 2, additional comprising:
said connection to communicate said electric signal stored in said electronic comprises a transceiver engaged with said housing; and
said transceiver wirelessly communicating with said remote communicating device.

5. The equine condition monitoring apparatus of claim 1, wherein said skin of said horse is positioned on a bottom surface of a tail of said horse adjacent the dock;
said housing having a recessed area at a central portion on said engagement side of said housing;
said at least one sensor being located in said central portion;
said flexible member having two portions, each said portion extending to distal ends from first ends engaged with opposing sides of said housing; and
cooperative fasteners positioned at said distal ends of said two portions to removably connect said two portions to a top surface of said tail opposite said bottom surface of said tail.

6. The equine condition monitoring apparatus of claim 4, wherein said skin of said horse is positioned on a bottom surface of a tail of said horse adjacent the dock;
said housing having a recessed area at a central portion on said engagement side of said housing;
said at least one sensor being located in said central portion;
said flexible member having two portions each said portion extending to distal ends from first ends engaged with opposing sides of said housing;
cooperative fasteners positioned at said distal ends of said two portions to removably connect said two portions to a top surface of said tail opposite said bottom surface of said tail;
an antenna positioned adjacent one of said distal ends; and
said antenna operatively connected with a lead to one or both of said transceiver and said terrestrial location sensor, where by communications transmitted by said transceiver emanate from said antenna and electronic signals employed by said terrestrial location sensor are received by said antenna and communicated to said terrestrial location sensor.

7. The equine condition monitoring apparatus of claim 6, wherein said terrestrial location sensor employs one or a combination of GPS satellite signals, cellular tower signals, or locally positioned beacon signals, communicated from said antenna, to determine a terrestrial location.

8. The equine condition monitoring apparatus of claim 3, wherein said skin of said horse is positioned on a bottom surface of a tongue in a mouth of said horse;
said housing having a recessed area at a central portion on said engagement side of said housing;
said at least one sensor being located in said central portion;
said flexible member having two portions each said portion extending to distal ends from first ends engaged with opposing sides of said housing;
cooperative fasteners positioned at said distal ends of said two portions to removably connect said two portions in an engagement upon a surface on the jaw of said horse exterior to said mouth;
an antenna positioned adjacent one of said distal ends adjacent said engagement; and
said antenna operatively connected with a lead to one or both of said transceiver and said terrestrial location sensor, where by communications transmitted by said transceiver emanate from said antenna and electronic signals employed by said terrestrial location sensor are received by said antenna and communicated to said terrestrial location sensor.

9. The equine condition monitoring apparatus of claim 5, wherein said at least one sensor is one or a combination of sensors from a group of sensors configured for sensing physiological characteristics including oxygen saturation, heart rate, breathing rate, body temperature, and fluid contents of saliva.

10. The equine condition monitoring apparatus of claim 6, wherein said at least one sensor is one or a combination of sensors from a group of sensors configured for sensing physiological characteristics including oxygen saturation, heart rate, breathing rate, body temperature, and fluid contents of saliva.

11. The equine condition monitoring apparatus of claim 7, wherein said at least one sensor is one or a combination of sensors from a group of sensors configured for sensing physiological characteristics including oxygen saturation, heart rate, breathing rate, body temperature, and fluid contents of saliva.

12. The equine condition monitoring apparatus of claim 8, wherein said at least one sensor is one or a combination of sensors from a group of sensors configured for sensing physiological characteristics including oxygen saturation, heart rate, breathing rate, body temperature, and fluid contents of saliva.

13. A method for monitoring physiological characteristics of a horse, comprising:
positioning at least one sensor adjacent to or in contact with a position on the body of a horse;
having said horse exercise on a track for a duration of time;
capturing an electric signal output by said at least on sensor correlating to a physiological characteristic of said horse during said duration of time;
storing a said electric signal from said one sensor in electronic memory;
employing software adapted to employ said electric signal to graphically depict said physiological characteristic represented by said electronic signal in a correlation to any portion of said duration of time said horse exercises on said track.

14. The method of claim 13, additionally comprising:
positioning a terrestrial location sensor positioned on said horse;
communicating a location signal output by said terrestrial location sensor to said electronic memory; and
including a location of said horse upon said track with depict said physiological characteristic represented by said electronic signal in a correlation to any portion of said duration of time said horse exercises on said track.

15. The method of claim 14, additionally comprising:
employing said location signal to calculate a speed of said horse at positions upon said track during any moment during said duration of time.
